(19) United States
(12) Patent Application Publication
(10) Pub. No.: US 2020/0329684 A1
(43) Pub. Date: Oct. 22, 2020

(54) METHOD FOR OBTAINING RETINAL DEGENERATION (rd) MODEL MOUSE HAVING LOW RISK OF CAUSING RETINAL DETACHMENT

(71) Applicants: Nippon Medical School Foundation, Tokyo (JP); Teika Pharmaceutical Co., Ltd., Toyama (JP)

(72) Inventors: Takashi Okada, Tokyo (JP); Tsutomu Igarashi, Tokyo (JP); Asaka Shiozawa, Toyama-shi (JP)

(21) Appl. No.: 16/846,718

(22) Filed: Apr. 13, 2020

(30) Foreign Application Priority Data
Apr. 17, 2019 (JP) 2019-078847

(51) Int. Cl. A01K 67/027

(52) U.S. Cl. CPC A01K 67/027; A01K 2267/03; A01K 2227/105

(57) ABSTRACT

Retinal detachment sometimes occurs during the aging process of retinal degeneration 6 model mouse (rd6 model mouse). The present invention provides a method for obtaining a retinal degeneration (rd) model mouse having a low risk of causing retinal detachment, including: acquiring tissue data about a retinal tissue in an rd model mouse by using a retinal examination means; determining whether or not the tissue data meets an evaluation criterion; and selecting the rd model mouse, the tissue data of which meets the evaluation criterion.

### TECHNICAL FIELD

The present invention relates to a method for obtaining a retinal degeneration (rd) model mouse having a low risk of causing retinal detachment.

### BACKGROUND ART

Representative examples of a cause for visual loss in Japan include glaucoma and pigmentary retinopathy. In some patients with glaucoma, the pathology advances while their intraocular pressure is adequately controlled. In addition, pigmentary retinopathy has a variety of causative genes. To date, there has thus been no effective treatment protocol. This necessitates treatment for protecting a retinal tissue, in particular, retinal ganglion cells, photoreceptor cells, and retinal pigment epithelial cells as they are.

Pigmentary retinopathy has been studied using a pigmentary retinopathy mouse model. Various model mice with different causative genes exist as the pigmentary retinopathy mouse model. The pathology of retinal degeneration 6 model mouse (rd6 model mouse) progresses more gradually than that of the other model mice, said a report.

A report (Hawes N L, et al., Investigative Ophthalmology & Visual Science September 2000, Vol. 41, 3149-3157) discloses that the rd6 model mouse has a genetic mutation in the gene of Membrane-type frizzled-related protein (Mfrp) which is expressed in retinal pigment epithelium at a high level; uniform white spots on the fundus are recognized at the age of 8 weeks; and an apparent decrease in the number of cells in the outer nuclear layer (ONL) is observed at the age of 3 months.

### SUMMARY OF INVENTION

### Technical Problem

Because the pathology of the rd6 mice advances more gradually than the other model mice, the rd6 mice have been used to yield reliable experimental results. The present inventors, however, have found that the experimental results are unreliable during research on pigmentary retinopathy using the rd6 mice. Detailed analysis has revealed that in the rd6 mice, retinal detachment has sometimes occurred during the aging process.

### Solution to Problem

The present inventors have conducted intensive research and, as a result, have elucidated that retinal detachment sometimes occurs by a specific aging time point; rd6 mice having no occurrence of retinal detachment by the specific aging time point are used for research on pigmentary retinopathy to yield reliable experimental results.

The invention relates to a method for obtaining a retinal degeneration (rd) model mouse having a low risk of causing retinal detachment, including:

acquiring tissue data about a retinal tissue in an rd model mouse by using a retinal examination means;

determining whether or not the tissue data meets an evaluation criterion; and

selecting the rd model mouse, the tissue data of which meets the evaluation criterion.

The invention allows for exclusion of model mice in which retinal detachment will occur. Thus, it is possible to obtain model mice used to yield reliable experimental results.

In addition, in the invention,

the retinal examination means may be at least one selected from the group consisting of a fundus camera, an optical coherence tomography apparatus, and an electroretinogram recording device.

In addition, in the invention,

when the retinal examination means is the fundus camera, the tissue data may be a fundus image;

when the retinal examination means is the optical coherence tomography apparatus, the tissue data may be a tomogram of the retinal tissue; and

when the retinal examination means is the electroretinogram recording device, the tissue data may be an electroretinogram.

In addition, in the invention,

(a) when the tissue data is the fundus image, the evaluation criterion may be that a retinal vessel does not look like a white line,

(b) when the tissue data is the tomogram of the retinal tissue, the evaluation criterion may be at least one selected from the group consisting of the following (1), (2), and (3):
(1) no occurrence of retinal detachment;
(2) no significant difference when a thickness of outer retinal layer is compared to a reference thickness; and
(3) the thickness of outer retinal layer being the reference thickness or more, and

(c) when the tissue data is the electroretinogram, the evaluation criterion may be the following (I) and/or (II):
(I) no significant difference when a b-wave amplitude is compared to a reference b-wave amplitude; and
(II) the b-wave amplitude being the reference b-wave amplitude or more.

In addition, in the invention, the reference may be a standard based on tissue data beforehand acquired from an rd model mouse having no occurrence of retinal detachment.

In addition, in the invention, the rd model mouse may be a 4- to 15-week-old rd model mouse.

In addition, in the invention, the rd model mouse may be an rd6 model mouse.

### BRIEF DESCRIPTION OF DRAWINGS

FIG. 1 shows tomograms of retinal tissues acquired from a (wild-type) C57BL/6 mouse at the age of 4 weeks, 6 weeks, and 11 weeks.

FIG. 2 shows tomograms of retinal tissues acquired from an rd6 ctrl mouse at the age of 4 weeks, 6 weeks, and 11 weeks.

FIG. 3 shows tomograms of retinal tissues acquired from an rd6 RD mouse at the age of 4 weeks, 6 weeks, and 11 weeks.

FIG. 4 shows the results of comparing the thicknesses of outer retinal layer among the wild-type, the rd6 ctrl, and the rd6 RD mice.

FIG. 5 shows the results of statistically analyzing the thicknesses of outer retinal layer among the wild-type, the rd6 ctrl, and the rd6 RD mice.

FIG. 6 shows electroretinograms (ERG) in wild-type, rd6 ctrl, and rd6 RD mice at the age of 5 weeks.

FIG. 7 shows ERGs in the wild-type, rd6 ctrl, and rd6 RD mice at the age of 10 weeks.

FIG. 8 shows the results of statistically analyzing the b-wave amplitudes of the ERGs among the wild-type, the rd6 ctrl, and the rd6 RD mice at the age of 5 weeks.

FIG. 9 shows the results of statistically analyzing the b-wave amplitudes of the ERGs among the wild-type, the rd6 ctrl, and the rd6 RD mice at the age of 10 weeks.

FIG. 10 shows fundus retinal vessel images (photographs) of wild-type, rd6 ctrl, and rd6 RD mice.

### DESCRIPTION OF EMBODIMENTS

### Overview and Definitions

Hereinafter, embodiments of the invention will be described in detail. Note that repeated descriptions of the same content are omitted so as to avoid redundancy.

For convenience, specific terms used in the present application are herein provided. Unless otherwise defined, all technical and scientific terms used herein have the same meaning as generally understood by those skilled in the art of the invention. Unless otherwise indicated in the text, a singular article “a”, “an”, or “the” includes plural references.

Numerical ranges and parameters indicated herein involve approximate values. However, the numbers designated in specific Examples are described as accurate as possible. Nevertheless, each number contains an intrinsic unavoidable specific error occurring with a standard deviation of values measured in respective tests. In addition, the term “about” used herein generally means 10%, 5%, 1%, or 0.5% of each prescribed value or range. Alternatively, the term “about” means that the number is within an acceptable standard error when considered by those skilled in the art.

Retinal Detachment

Retinal detachment is a disease in which the retina, including nine neuroretinal layers and one retinal pigment epithelial cell layer present on the deepest side of an eyeball, is detached. In the disease, visual acuity is lowered markedly, and there is thus a risk of visual loss.

Retinal Degeneration (rd) Model Mouse

Examples of the retinal degeneration (rd) model mouse include various model mice with different causative genes. Here, rd6 model mice are a model in which the pathology advances more gradually than the other models. A report discloses that this model has a genetic mutation in the gene of Membrane-type frizzled-related protein (Mfrp) which is highly expressed in retinal pigment epithelium; uniform white spots on the fundus are recognized at the age of 8 weeks; and an apparent decrease in the number of cells in the outer nuclear layer (ONL) is observed at the age of 3 months.

### EMBODIMENTS

An embodiment of the invention provides

a method for obtaining a retinal degeneration (rd) model mouse having a low risk of causing retinal detachment, including:

acquiring tissue data about a retinal tissue in an rd model mouse by using a retinal examination means;

determining whether or not the tissue data meets an evaluation criterion; and

selecting the rd model mouse, the tissue data of which meets the evaluation criterion.

The rd model mouse, the tissue data of which meets the evaluation criterion, has a low risk of causing retinal detachment as demonstrated in the below-described Examples.

In this embodiment, the wording “low risk of causing retinal detachment” means that individual mice or a mouse group will have no occurrence of retinal detachment, which substantially affects experimental results, under regular aging conditions by the mouse age appropriate for the experiment. The degree of “retinal detachment, which substantially affects experimental results” may be altered depending on the experiment. In another embodiment, the “low risk of causing retinal detachment” may mean “no risk of causing retinal detachment”. Whether or not retinal detachment occurs can be easily judged by the skilled artisan. The technology disclosed in JP Patent Publication NO. 2017-127397 may be used to determine whether or not retinal detachment occurs. Also, it is possible to determine whether or not the risk of causing retinal detachment is low or whether or not there is a risk of causing retinal detachment.

The retinal examination means is not particularly limited as long as acquired tissue data can be used to distinguish between an rd model mouse having a low risk of causing retinal detachment and an rd model mouse having a high risk of causing retinal detachment or in which retinal detachment has occurred. Examples include visual electrophysiological testing devices such as a fundus camera, a tomographic system (e.g., an optical coherence tomography apparatus), and an electroretinogram recording device. One of the retinal examination means may be used singly, or multiple retinal examination means may be used in combination. When multiple retinal examination means are used in combination, criteria fit for each retinal examination means should be provided. The retinal examination means may be modified depending on the age of the rd model mouse.

Examples of the tissue data include images (e.g., photographs), numbers, and graphs. When the tissue data include an image(s) or a graph(s), the tissue data and a reference may be compared visually or by using a computer. When the tissue data is a fundus image(s), it is preferable that the fundus image is a color image. When the retinal examination means is an optical coherence tomography apparatus, the tissue data is typically a tomogram(s) (image(s)) of the retinal tissue. When the retinal examination means is an electroretinogram recording device, the tissue data is typically an electroretinogram(s) (graph(s)).

The evaluation criterion may be not only one evaluation criterion, but also a combination of evaluation criteria.

When the tissue data is a fundus image(s), the evaluation criterion may be that a retinal vessel does not look like a white line.

When the tissue data is a tomogram(s) of the retinal tissue, the evaluation criterion may be at least one selected from the group consisting of the following (1), (2), and (3):

(1) no occurrence of retinal detachment;

(2) no significant difference when a thickness of outer retinal layer is compared to a reference thickness; and

(3) the thickness of outer retinal layer being the reference thickness or more.

When the tissue data is an electroretinogram(s), the evaluation criterion may be the following (I) and/or (II):

(I) no significant difference when a b-wave amplitude is compared to a reference b-wave amplitude; and

(II) the b-wave amplitude being the reference b-wave amplitude or more.

Meanwhile, the reference may be a standard based on tissue data beforehand acquired from an rd model mouse having no occurrence of retinal detachment by the mouse age appropriate for the experiment. The tissue data used for the reference may be data from one or more tissues or tissue data (e.g., the mean and standard error) obtained by statistically processing multiple tissue data. Whether a retinal vessel looks like a white line, the presence of retinal detachment, the thickness of outer retinal layer, and the b-wave amplitude may be determined based on knowledge of specialists, by referring to a reference, or further by using a computer.

An rd model mouse (mice), tissue data of which meets the evaluation criterion, is selectively included. An rd model mouse (mice), tissue data of which fails to meet the evaluation criterion, is selectively excluded. In this way, an rd model mouse (mice) having a low risk of causing retinal detachment can be selectively obtained.

The rd model mice used in this embodiment may be 4- to 11-week-old rd model mice. The age may be any of 4, 5, 6, 7, 8, 9, 10, 11, 12, 13, 14 or 15 weeks or may be in a range between any two of the above points (e.g., between 5 and 7 weeks). In some experiments, an rd model mouse (mice) at the age of 16 weeks or more may be used.

The method according to this embodiment may be used to obtain an rd model mouse having a low risk of causing retinal detachment. Here, this method may be repeated multiple times. For instance, the method according to this embodiment may be used to obtain a 4-week-old rd model mouse having a low risk of causing retinal detachment. Two weeks after that, the method according to this embodiment may be repeated on this rd model mouse (i.e., a 6-week-old rd model mouse). This makes it possible to obtain an rd model mouse having a lower risk of causing retinal detachment.

### EXAMPLES

### Experimental Example 1: Tomographic Time-Course Observation of Retinal Tissue in (Wild-Type) C57BL/6 Mouse

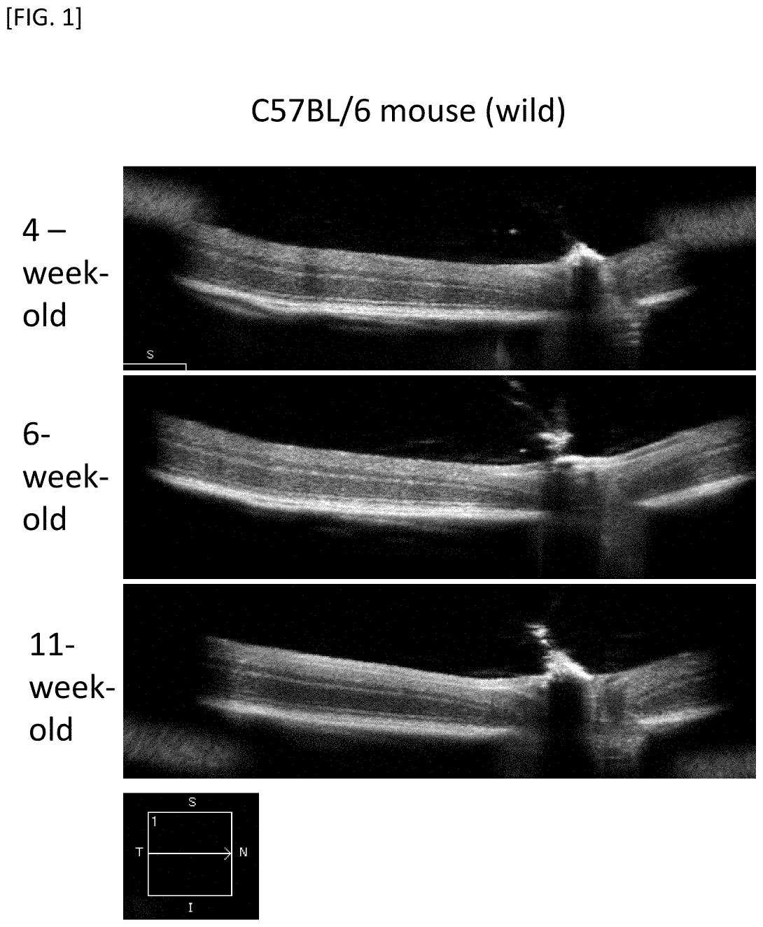

Tomograms of retinal tissues from a (wild-type, male) C57BL/6 mouse (CLEA Japan, Inc.) were recorded over time. The mouse was anesthetized while intraperitoneally given ketamine/xylazine (100 mg/kg and 10 mg/kg) and given oxybuprocaine hydrochloride (0.4% benoxil eye drops, Santen Pharmaceutical Co., Ltd.) to an eye. The tomograms of retinal tissues were acquired using an optical coherence tomography apparatus (by OCT: optical coherence tomography) (Cirrus HD-OCT, Carl Zeiss Meditec AG). The OCT apparatus equipped with a SuperField NC (Volk Optical, Inc.) was used to capture an image. Tomograms of fundus retinal tissues in the following experiments were optic nerve-containing tomograms. When OCT was applied to a C57BL/6 mouse, Opegan (SEIKAGAKU CORPORATION) and a cover glass (Matsunami Glass Ind., Ltd.) were used to manufacture a planoconcave lens, which was then mounted on an eye of the C57BL/6 mouse having the OCT lens to be attached. When the (wild-type) C57BL/6 mouse was at the age of 4 weeks, 6 weeks, or 11 weeks, tomograms were acquired. FIG. 1 shows the observation results. The respective layers were clearly observed in the (wild-type) C57BL/6 mice.

### Experimental Example 2: Tomographic Time-Course Observation of Retinal Tissue in Retinal Degeneration 6 (Rd6) C57BL/6 Mice

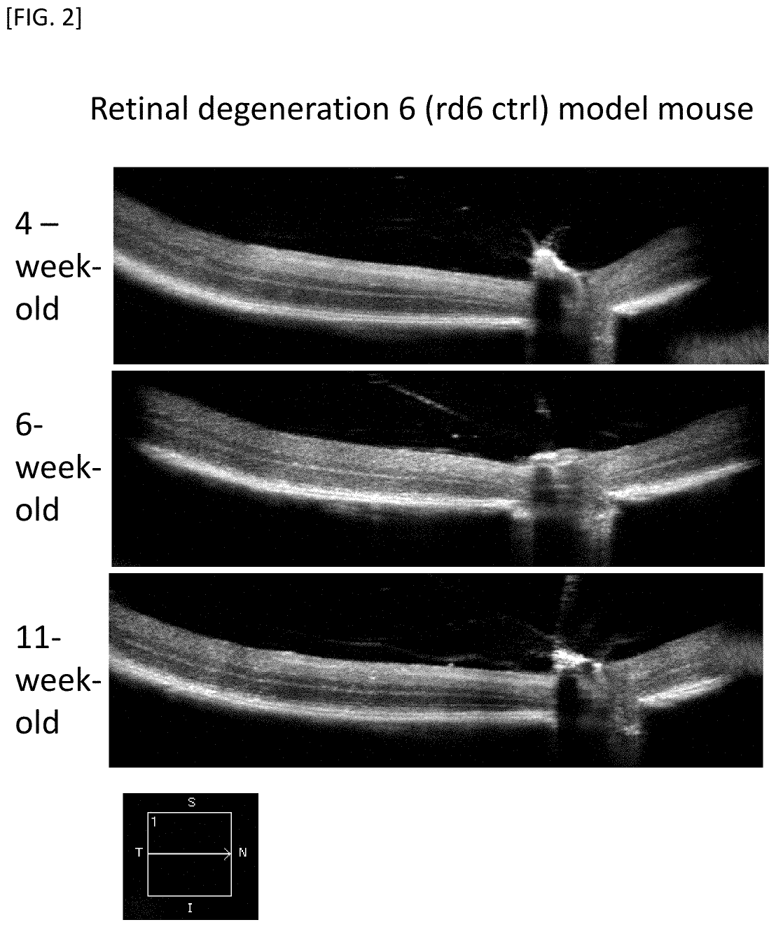

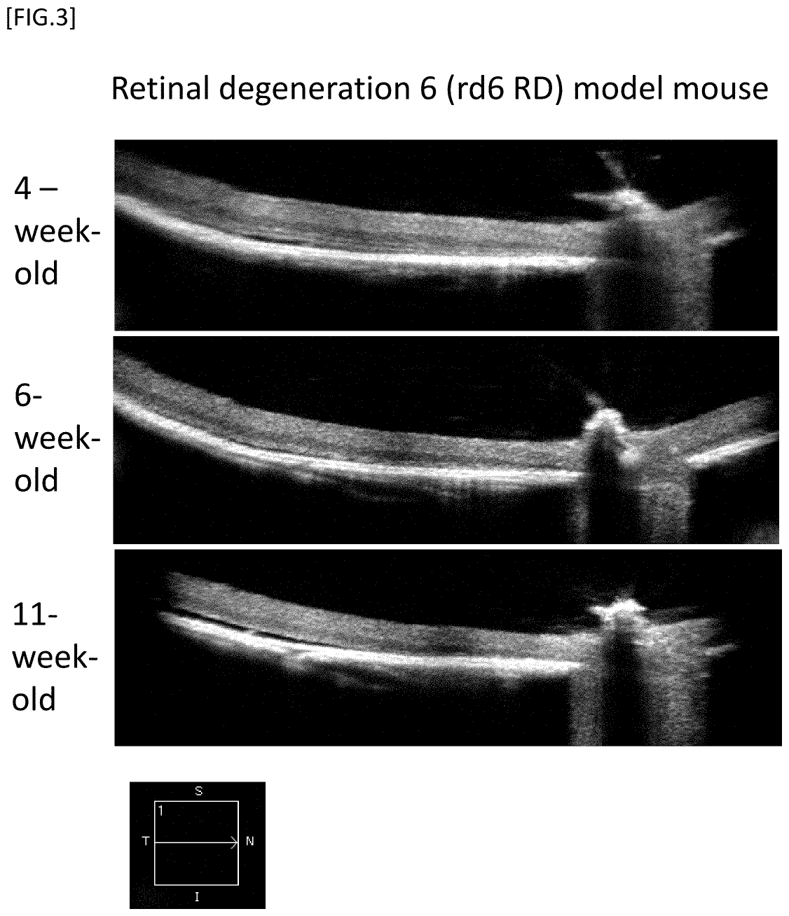

Tomograms of retinal tissues in two retinal degeneration 6 (rd6) model mice (The Jackson Laboratory) were recorded over time. The experiment was conducted in accordance with the protocol described in Experimental Example 1. FIGS. 2 and 3 show the observation results.

FIG. 2 shows tomograms of retinal tissues from one of the two rd6 mice. The tomograms of retinal tissues of the rd6 mouse as shown in FIG. 2 had an unclear ellipsoid zone (EZ). However, the outer nuclear layer (ONL) was observed, and no retinal detachment was recorded. The rd6 mouse without recoded retinal detachment is hereinafter referred as rd6 ctrl mouse.

FIG. 3 shows tomograms of retinal tissues from the other rd6 mouse. In this rd6 mouse at the age of 4 weeks, retinal detachment was observed, and a very thin ONL was recorded. In this rd6 mouse at the age of 6 weeks, retinal detachment progression was observed, and no ONL was recorded. In this rd6 mouse at the age of 11 weeks, severe retinal detachment was observed. The rd6 mouse with recoded retinal detachment is hereinafter referred as rd6 RD mouse.

### Experimental Example 3: Comparison in Outer Retinal Layer Among Wild-Type Mouse and Respective Rd6 Mice

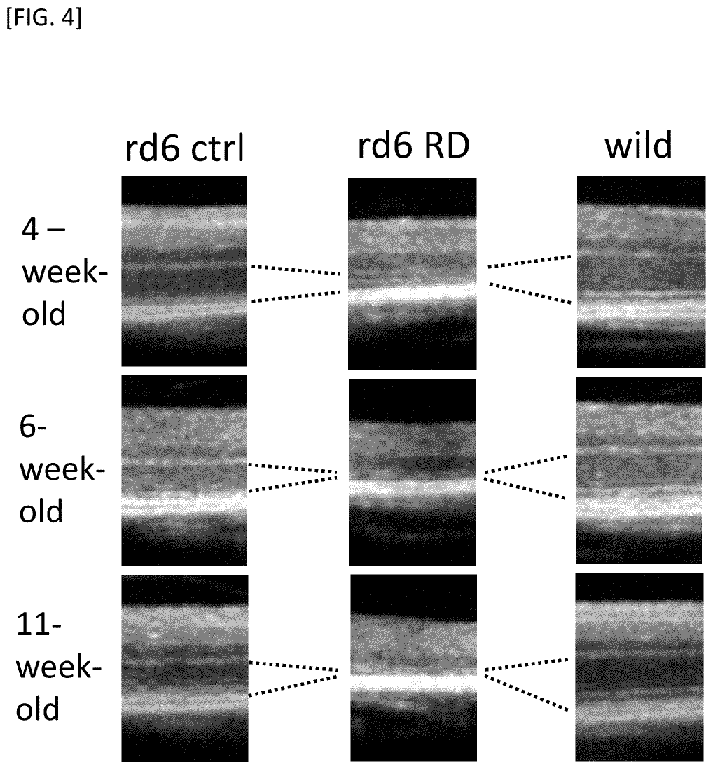

The thicknesses of outer retinal layer among the wild-type, the rd6 ctrl, and the rd6 RD mice were compared. FIG. 4 shows tomograms of outer retinal layer in the respective mice. Dashed lines are used to compare the thicknesses of outer retinal layer among the mice. It was revealed that the thickness of outer retinal layer in the rd6 RD mouse was markedly thinner than those of the rd6 ctrl mouse and the wild-type mouse.

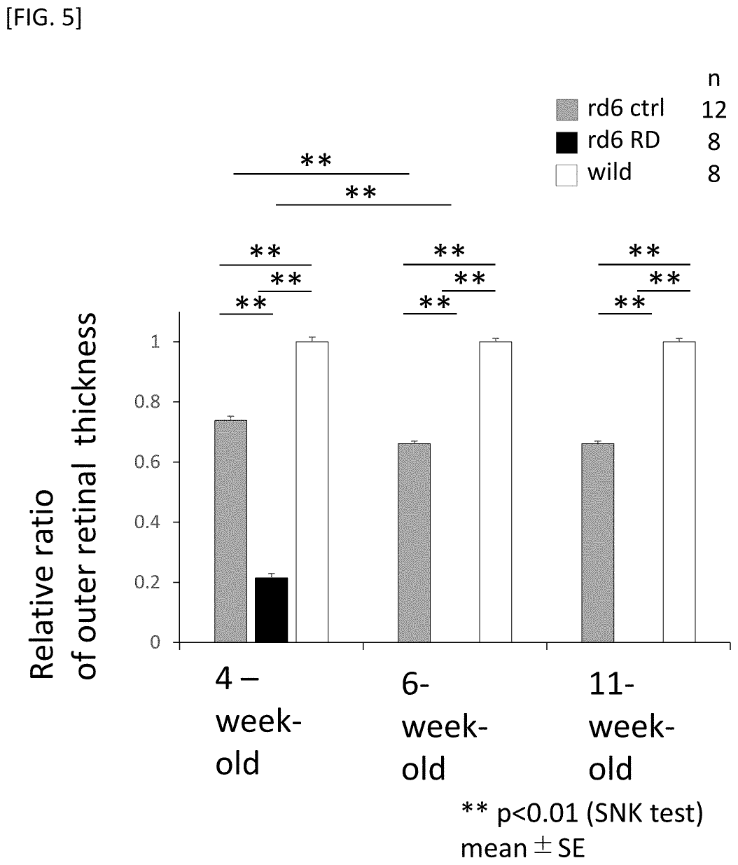

The thicknesses of outer retinal layer among the wild-type, the rd6 ctrl, and the rd6 RD mice were statistical analyzed. In the statistical analysis, 8 eyes of wild-type mice, 12 eyes of rd6 ctrl mice, and 8 eyes of rd6 RD mice were used. FIG. 5 shows the results. FIG. 5 clearly demonstrated that the outer retinal layer of the rd6 RD mouse became markedly thinner.

### Experimental Example 4: Electroretinograms (ERGs) of Wild-Type Mouse and Respective Rd6 Mice

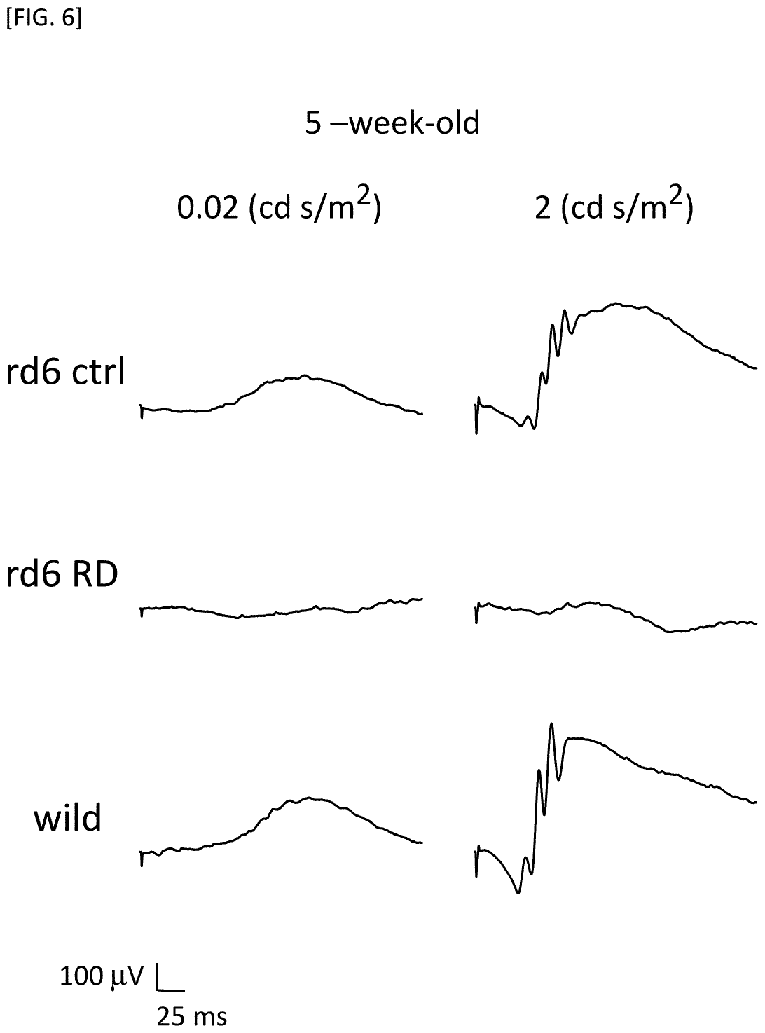

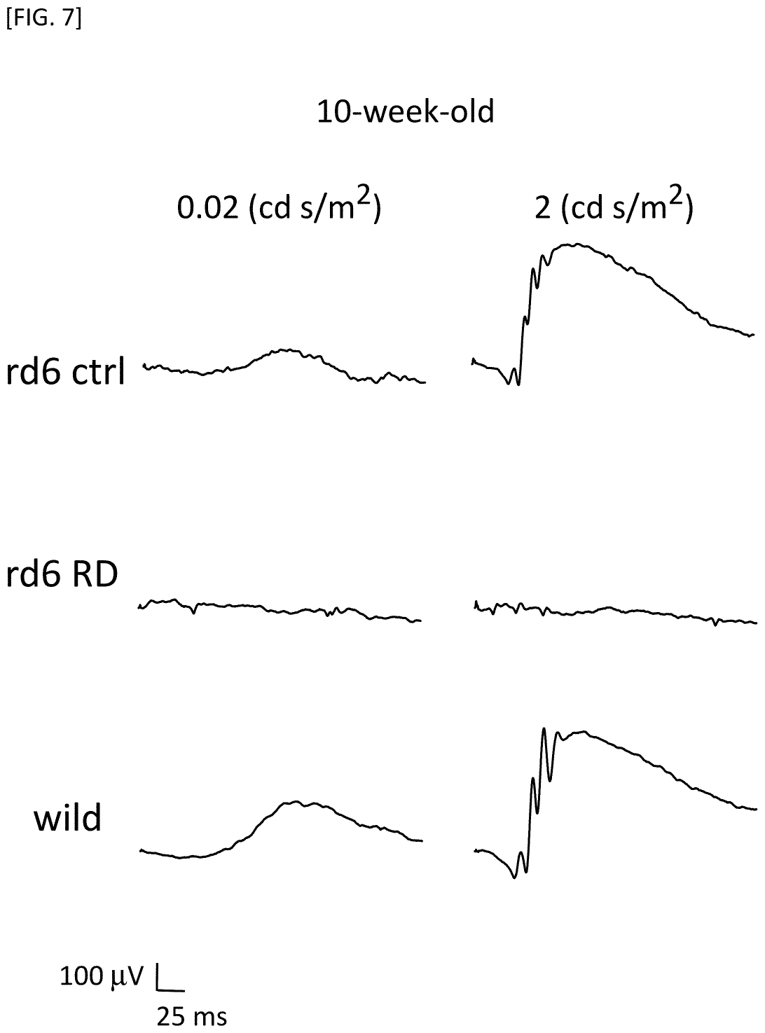

Electroretinograms of wild-type, rd6 ctrl, and rd6 RD mice were acquired. The ERGs were recorded using an LS-W (MAYO Corporation) as a photo-stimulator, a PowerLab 2/26 (ADInstruments) as an A/D converter, and a Bio Amp ML132 (ADInstruments) as an amplifier. The stimulus intensity in the ERGs was set to 0.02 cd s/m² or 2.0 cd s/m². FIG. 6 shows ERGs of the mice at the age of 5 weeks, and FIG. 7 shows ERGs of the mice at the age of 10 weeks. In the rd6 RD mouse at the age of 5 weeks, a slight b-wave was just recorded when the stimulus intensity was 2.0 cd s/m². At the age of 10 weeks, no response was recorded when the stimulus intensity was either 0.02 or 2.0 cd s/m².

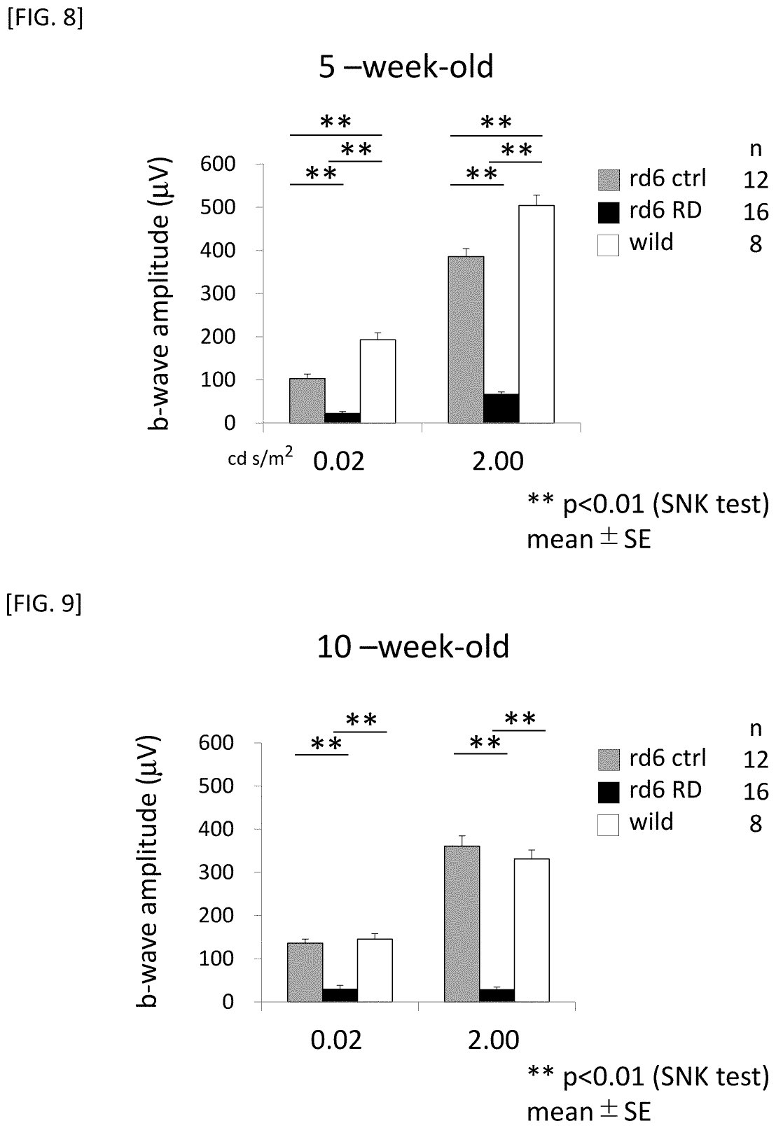

The b-wave amplitudes in the wild-type, the rd6 ctrl, and the rd6 RD mice were statistical analyzed. In the statistical analysis, 8 eyes of wild-type mice, 12 eyes of rd6 ctrl mice, and 8 eyes of rd6 RD mice were used. FIG. 8 shows the results in the respective mice at the age of 5 weeks, and FIG. 9 shows the results in the respective mice at the age of 10 weeks. FIGS. 8 and 9 clearly demonstrated that the b-wave amplitudes of ERGs in the rd6 RD mouse decreased markedly.

### Experimental Example 5: Examination of Retinal Vessels in Wild-Type Mouse and Respective rd6 Mice

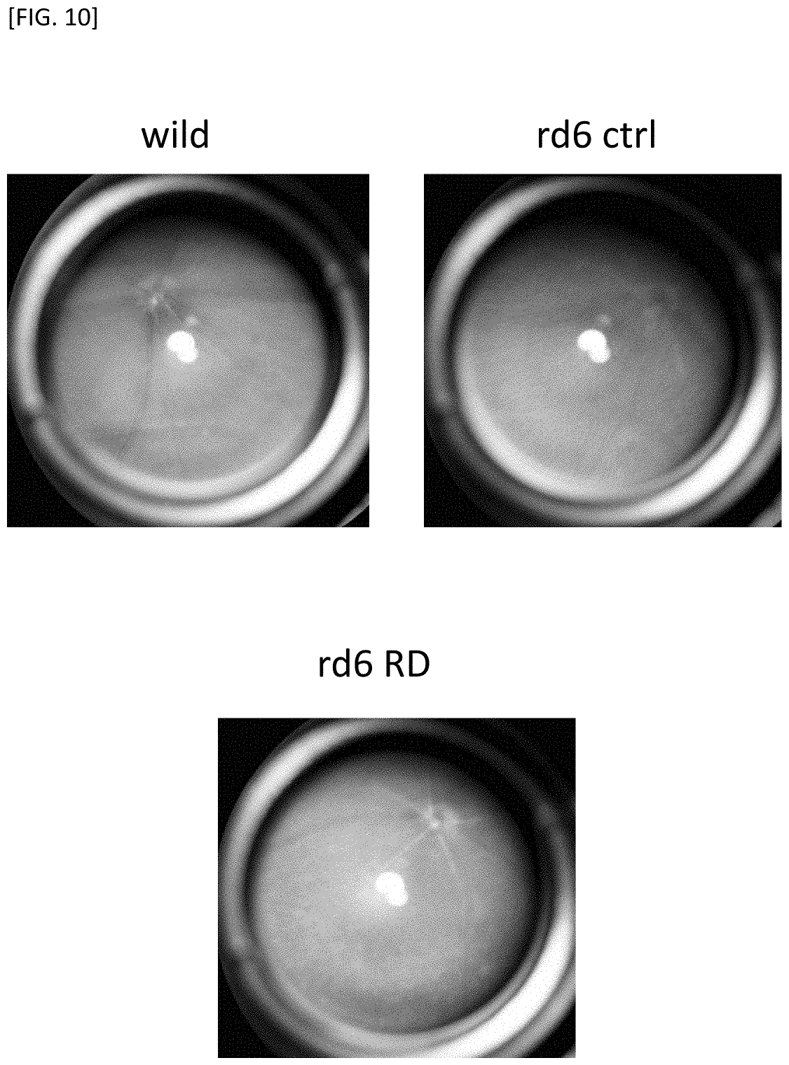

Images of fundus retinal vessels in wild-type, rd6 ctrl, and rd6 RD mice at the age of 14 weeks were acquired. The images were captured using a Genesis-Df (Kowa Company, Ltd.). FIG. 10 shows the results. In the wild-type and the rd6 ctrl mice, no blood vessel abnormality was seen. In the rd6 RD mouse, however, a blood vessel(s) looked like a white line. As reported in Hawes et al., the rd6 ctrl mouse had uniform white spots. By contrast, the rd6 RD mouse had whitish patches over the entire fundus.

1. A method for obtaining a retinal degeneration (rd) model mouse having a low risk of causing retinal detachment, comprising:
acquiring tissue data about a retinal tissue in an rd model mouse by using a retinal examination means;
determining whether or not the tissue data meets an evaluation criterion; and
selecting the rd model mouse, the tissue data of which meets the evaluation criterion.

2. The method according to claim 1, wherein the retinal examination means is at least one selected from the group consisting of a fundus camera, an optical coherence tomography apparatus, and an electroretinogram recording device.

3. The method according to claim 2, wherein when the retinal examination means is the fundus camera, the tissue data is a fundus image;
when the retinal examination means is the optical coherence tomography apparatus, the tissue data is a tomogram of the retinal tissue; and
when the retinal examination means is the electroretinogram recording device, the tissue data is an electroretinogram.

4. The method according to claim 3, wherein
(a) when the tissue data is the fundus image, the evaluation criterion is that a retinal vessel does not look like a white line,
(b) when the tissue data is the tomogram of the retinal tissue, the evaluation criterion is at least one selected from the group consisting of the following (1), (2), and (3):
(1) no occurrence of retinal detachment;
(2) no significant difference when a thickness of outer retinal layer is compared to a reference thickness; and
(3) the thickness of outer retinal layer being the reference thickness or more, and
(c) when the tissue data is the electroretinogram, the evaluation criterion is the following (I) and/or (II):
(I) no significant difference when a b-wave amplitude is compared to a reference b-wave amplitude; and
(II) the b-wave amplitude being the reference b-wave amplitude or more.

5. The method according to claim 4, wherein the reference is a standard based on tissue data beforehand acquired from an rd model mouse having no occurrence of retinal detachment.

6. The method according to claim 1, wherein the rd model mouse is a 4- to 15-week-old rd model mouse.

7. The method according to claim 1, wherein the rd model mouse is an rd6 model mouse.
